(19) United States
(12) Patent Application Publication
(10) Pub. No.: US 2022/0362460 A1
(43) Pub. Date: Nov. 17, 2022

(54) INFUSION APPARATUS

(71) Applicants: Inventec Appliances (Pudong) Corporation, Shanghai (CN); Inventec Appliances (Shanghai) Co., Ltd., Shanghai (CN); Inventec Appliances Corp., New Taipei City (TW)

(72) Inventors: Shou-tien YIN, New Taipei City (TW); Hon-wing AU, New Taipei City (TW); Wei-cheng LIN, New Taipei City (TW); Chi-lin LEE, New Taipei City (TW); Yi-yu LEE, New Taipei City (TW)

(21) Appl. No.: 17/618,896

(22) PCT Filed: Jun. 14, 2019
(86) PCT No.: PCT/CN2019/091344

§ 371 Date: Dec. 14, 2021

(51) Int. Cl. A61M 5/168

(52) U.S. Cl. CPC A61M 5/16813

(57) ABSTRACT

An infusion apparatus includes a fixture body, a tube socket and a buckling device. The fixture body has a first and a second ends along a first direction. The fixture body has a through hole. Two first protruding pieces are located at the second end along a second direction perpendicular with the first direction. The fixture body penetrates through and is slidable relative with the tube socket which has two grooves along the second direction. The buckling device includes a machine body and two hooking structures. The machine body has a mounting hole. The hooking structures are disposed at opposite sides of the mounting hole and respectively include a hook and a second protruding piece. The hooks buckle within the grooves. A shortest distance between the second protruding pieces is less than a longest distance between the first protruding pieces.

### CROSS-REFERENCE TO RELATED APPLICATION

This application is a U.S. National Phase application of PCT/CN2019/091344 filed Jun. 14, 2019, which is herein incorporated by reference.

### BACKGROUND

### Technical Field

The present disclosure relates to infusion apparatus.

### Description of Related Art

In a society which is growing to aging and longevity, the demand for medical equipment is rapidly increasing. Therefore, the quality of medical equipment has also become a concern. The reliability and safety of medical equipment are undoubtedly an important issue in the industry.

In general, it is a common practice in medical procedures to use infusion apparatus to deliver medical fluids to patients. However, during the operation of the infusion pump, every step must be handled very carefully to avoid unexpected fluids from flowing into the patient's body.

### SUMMARY

A technical aspect of the present disclosure is to provide an infusion apparatus, which can make the process to install the infusion pipe to the buckling device or to separate the infusion pipe from the buckling device become more stable.

According to an embodiment of the present disclosure, an infusion apparatus includes a fixture body, a tube socket and a buckling device. The fixture body has a first end and a second end opposite to the first end along a first direction. The fixture body has a first through hole extending along the first direction. The first through hole includes a first subsidiary through hole and a second subsidiary through hole communicating with the first subsidiary through hole. The first subsidiary through hole and the second subsidiary through hole are arranged along the first direction. A first width of the first subsidiary through hole along a second direction is larger than a second width of the second subsidiary through hole along the second direction. The fixture body is disposed with a pair of first protruding pieces. The first protruding pieces are located at two opposite sides of the second end along the second direction. The second direction is perpendicular with the first direction. The tube socket has a second through hole and a third through hole. The fixture body penetrates through the second through hole and is slidable relative to the tube socket along the first direction. The tube socket further has a pair of grooves located at two opposite sides of the tube socket along the second direction. When the tube socket slides relative to the fixture body, the third through hole is able to switch between a first position corresponding to the first subsidiary through hole and a second position corresponding to the second subsidiary through hole. The buckling device includes a machine body and a pair of hooking structures. The machine body has a mounting hole configured to allow the fixture body and the tube socket to penetrate into along the first direction. The hooking structures are disposed on the machine body and located at two opposite sides of the mounting hole along the second direction. Each of the hooking structures has a hook and a second protruding piece. The hooks are configured to buckle within the grooves. A shortest distance between the second protruding pieces is less than a longest distance between the first protruding pieces.

In one or more embodiments of the present disclosure, the first subsidiary through hole is closer to the first end than the second subsidiary through hole to the first end.

In one or more embodiments of the present disclosure, a third width of the third through hole along the second direction is larger than the second width.

In one or more embodiments of the present disclosure, the first width is equal to the third width.

In one or more embodiments of the present disclosure, the first width of the first subsidiary through hole diminishes towards the second subsidiary through hole.

In one or more embodiments of the present disclosure, the first protruding pieces are symmetrical with each other.

In one or more embodiments of the present disclosure, each of the hooking structures has a pivotally connecting portion pivotally connected with the machine body. Each of the second protruding pieces is located between the corresponding pivotally connecting portion and the corresponding hook.

In one or more embodiments of the present disclosure, the buckling device further includes a pair of elastic elements respectively and elastically connected between the corresponding hooking structure and the machine body.

In one or more embodiments of the present disclosure, each of the second protruding pieces has a curved surface. The curved surfaces face to each other.

In one or more embodiments of the present disclosure, each of the first protruding pieces has a trapezoidal shape.

When compared with the prior art, the above-mentioned embodiments of the present disclosure have at least the following advantage:

(1) Since the fixture body and the first protruding pieces are symmetrical structures, when the user inserts the fixture body together with the first protruding pieces into the buckling device or pulls out the fixture body together with the first protruding pieces from the buckling device, the forces that the second protruding pieces exert on the fixture body when abutting against the first protruding pieces are also symmetrical. In this way, the process to install the infusion pipe to the buckling device or to separate the infusion pipe from the buckling device becomes more stable. In addition, the risk of damage to the fixture body, the first protruding pieces and the hooking structures due to uneven force exertion is also reduced.

(2) Before the infusion pipe is separated from the buckling device, the infusion pipe is already flattened within the second subsidiary through hole of the fixture body. Thus, the medical fluid is prevented from flowing through the infusion pipe. Moreover, since the operation described above is carried out in a mechanical manner, the reliability and safety of the operation can be effectively guaranteed.

### BRIEF DESCRIPTION OF THE DRAWINGS

The disclosure can be more fully understood by reading the following detailed description of the embodiments, with reference made to the accompanying drawings as follows:

FIG. 1 is a three-dimensional assembly diagram of a fixture body and a tube socket according to an embodiment of the present disclosure;

FIG. 2 is a partial sectional view of the fixture body and the tube socket of FIG. 1 penetrating into a buckling device, in which the first protruding pieces abut against the second protruding pieces;

FIG. 3 is a partial sectional view of the fixture body and the tube socket of FIG. 1 penetrating into a buckling device, in which the hooks of the hooking structures buckle within the grooves of the tube socket;

FIG. 4 is a partial sectional view of the fixture body and the tube socket of FIG. 1 being pulled from a buckling device, in which the third through hole of the tube socket corresponds to the second subsidiary through hole of the fixture body; and

FIG. 5 is a partial sectional view of the fixture body and the tube socket of FIG. 1 being pulled from a buckling device, in which the fixture body and the tube socket are already separated from the buckling device.

### DETAILED DESCRIPTION

Drawings will be used below to disclose embodiments of the present disclosure. For the sake of clear illustration, many practical details will be explained together in the description below. However, it is appreciated that the practical details should not be used to limit the claimed scope. In other words, in some embodiments of the present disclosure, the practical details are not essential. Moreover, for the sake of drawing simplification, some customary structures and elements in the drawings will be schematically shown in a simplified way. Wherever possible, the same reference numbers are used in the drawings and the description to refer to the same or like parts.

Unless otherwise defined, all terms (including technical and scientific terms) used herein have the same meanings as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

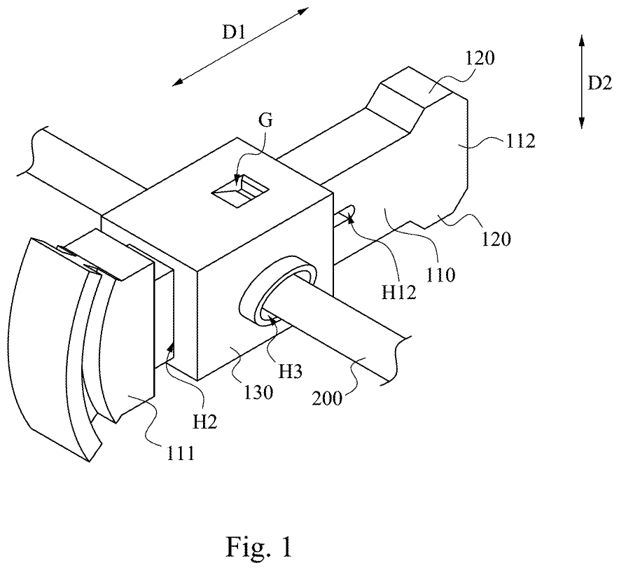

Reference is made to FIG. 1. FIG. 1 is a three-dimensional assembly diagram of a fixture body 110 and a tube socket 130 according to an embodiment of the present disclosure. In this embodiment, as shown in FIG. 1, the fixture body 110 and the tube socket 130 are suitable for an infusion pipe 200 to penetrate through. The tube socket 130 together with the infusion pipe 200 is slidable relative to the fixture body 110 along a first direction D1.

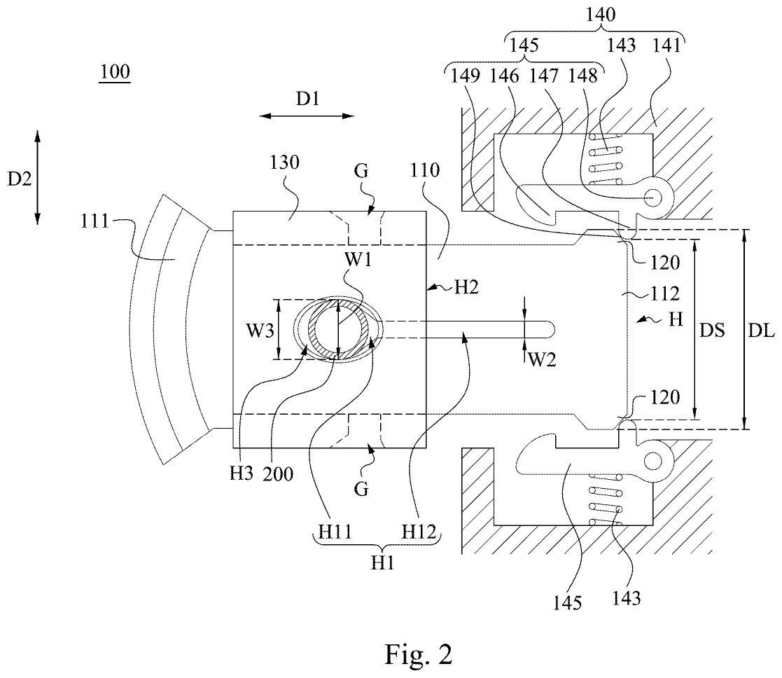

Reference is made to FIG. 2. FIG. 2 is a partial sectional view of the fixture body 110 and the tube socket 130 of FIG. 1 penetrating into a buckling device 140, in which the first protruding pieces 120 abut against the second protruding pieces 147. In this embodiment, as shown in FIGS. 1-2, an infusion apparatus 100 includes the fixture body 110, the tube socket 130 and a buckling device 140. The fixture body 110 has a first end 111 and a second end 112 opposite to the first end 111 along the first direction D1. The fixture body 110 has a first through hole H1. The first through hole H1 extends along the first direction D1. The first through hole H1 is suitable for the infusion pipe 200 to penetrate through. The fixture body 110 is disposed with a pair of first protruding pieces 120. The first protruding pieces 120 are located at two opposite sides of the second end 112 along a second direction D2. The second direction D2 is perpendicular with the first direction D1. The tube socket 130 has a second through hole H2. The fixture body 110 penetrates through the second through hole H2 and is slidable relative to the tube socket 130 along the first direction D1 as mentioned above. In other words, the tube socket 130 together with the infusion pipe 200 is slidable relative to the fixture body 110 along the first direction D1, and the infusion pipe 200 is slidable relative to the fixture body 110 along the first direction D1 within the first through hole H1. The tube socket 130 further has a pair of grooves G. The grooves G are located at two opposite sides of the tube socket 130 along the second direction D2. In this embodiment, the grooves G of the tube socket 130 are communicated with the second through hole H2. However, this does not intend to limit the present disclosure.

In addition, the buckling device 140 includes a machine body 141 and a pair of hooking structures 145. The machine body 141 has a mounting hole H. The mounting hole H is configured to allow the fixture body 110 and the tube socket 130 to penetrate into the machine body 141 along the first direction D1. The hooking structures 145 are disposed on the machine body 141 and located at two opposite sides of the mounting hole H along the second direction D2. This means the mounting hole H is located between the hooking structures 145. Each of the hooking structures 145 has a hook 146 and a second protruding piece 147. The hooks 146 are configured to buckle within the grooves G. A shortest distance DS between the second protruding pieces 147 is less than a longest distance DL between the first protruding pieces 120.

To be specific, as shown in FIG. 2, each of the hooking structures 145 has a pivotally connecting portion 148. The pivotally connecting portions 148 are pivotally connected with the machine body 141. Each of the second protruding pieces 147 is located between the corresponding pivotally connecting portion 148 and the corresponding hook 146. As mentioned above, the shortest distance DS between the second protruding pieces 147 is less than the longest distance DL between the first protruding pieces 120. Therefore, when the fixture body 110 and the tube socket 130 insert into the mounting hole H of the machine body 141 along the first direction D1, the first protruding pieces 120 abut against the second protruding pieces 147 of the hooking structures 145, such that the hooking structures 145 rotate about the pivotally connecting portions 148 relative to the machine body 141. Thus, the first protruding pieces 120 pass through the second protruding pieces 147 and insert into the machine body 141.

Furthermore, as shown in FIG. 2, each of the first protruding pieces 120 has a trapezoidal shape. Therefore, when the first protruding pieces 120 abut against the second protruding pieces 147 of the hooking structures 145, the inclined surface formed from the trapezoidal shape of each of the first protruding pieces 120 facilitates the first protruding pieces 120 to pass through the second protruding pieces 147. Moreover, each of the second protruding pieces 147 of the hooking structures 145 has a curved surface 149. The curved surfaces 149 face to each other. Therefore, when the hooking structures 145 rotate about the pivotally connecting portions 148 relative to the machine body 141, the curved surfaces 149 of the second protruding pieces 147 can be smoothly slidable with the inclined surfaces formed from the trapezoidal shapes of the first protruding pieces 120.

On the other hand, in this embodiment, as shown in FIG. 2, the buckling device 140 further includes a pair of elastic elements 143. The elastic elements 143 are respectively and elastically connected between the corresponding hooking structure 145 and the machine body 141. Therefore, after the hooking structures 145 rotate about the pivotally connecting portions 148 relative to the machine body 141, such that the first protruding pieces 120 pass through the second protruding pieces 147 and insert into the machine body 141, the elastic elements 143 can recover the position of the hooking structures 145. In practical applications, each of the elastic elements 143 can be a spring or a torsion spring. However, this does not intend to limit the present disclosure.

Structurally speaking, the first through hole H1 includes a first subsidiary through hole H11 and a second subsidiary through hole H12 communicating with the first subsidiary through hole H11. The first subsidiary through hole H11 and the second subsidiary through hole H12 are arranged along the first direction D1. The first subsidiary through hole H11 is closer to the first end 111 than the second subsidiary through hole H12 to the first end 111. A first width W1 of the first subsidiary through hole H11 along the second direction D2 is larger than a second width W2 of the second subsidiary through hole H12 along the second direction D2.

In addition, as shown in FIG. 2, the tube socket 130 has a third through hole H3. The third through hole H3 is suitable for the infusion pipe 200 to penetrate through. A third width W3 of the third through hole H3 of the tube socket 130 along the second direction D2 is larger than the second width W2 of the second subsidiary through hole H12. In practical applications, the first width W1 of the first subsidiary through hole H11 is equal to the third width W3 of the third through hole H3. However, this does not intend to limit the present disclosure.

When a user uses the infusion pipe 200 to penetrate through the tube socket 130 and the fixture body 110, the infusion pipe 200 penetrates through the third through hole H3 of the tube socket 130 and the first subsidiary through hole H11 of the fixture body 110. When the tube socket 130 slides relative to the fixture body 110, the third through hole H3 is able to switch between a first position corresponding to the first subsidiary through hole H11 and a second position corresponding to the second subsidiary through hole H12. This means the infusion pipe 200 is able to follow the third through hole H3 and switch between the first subsidiary through hole H11 and the second subsidiary through hole H12. Moreover, as shown in FIG. 2, the first width W1 of the first subsidiary through hole H11 diminishes towards the second subsidiary through hole H12, which facilitates the degree of smoothness when the infusion pipe 200 switches between the first subsidiary through hole H11 and the second subsidiary through hole H12.

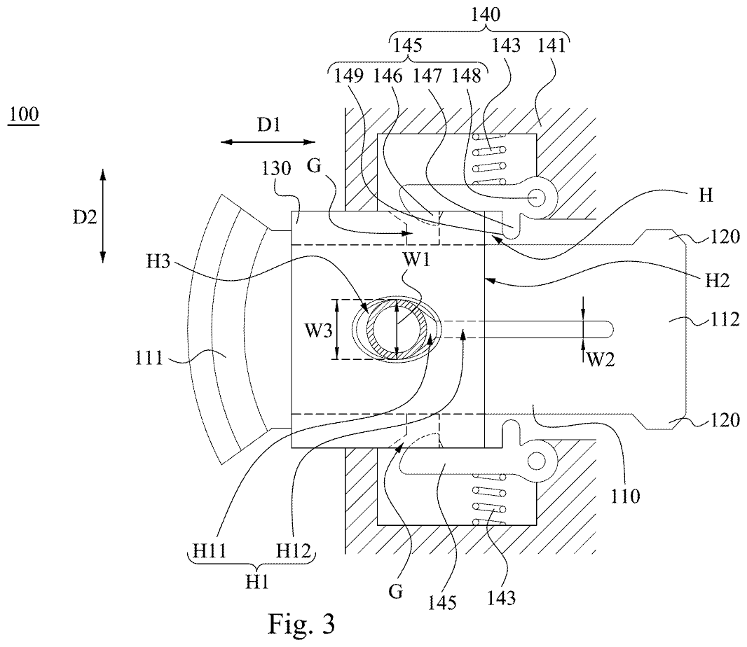

Reference is made to FIG. 3. FIG. 3 is a partial sectional view of the fixture body 110 and the tube socket 130 of FIG. 1 penetrating into a buckling device 140, in which the hooks 146 of the hooking structures 145 buckle within the grooves G of the tube socket 130. As shown in FIG. 3, after the first protruding pieces 120 pass through the second protruding pieces 147 of the hooking structures 145, the fixture body 110 and the tube socket 130, together with the infusion pipe 200, continue to insert into the mounting hole H of the machine body 141. Similarly, when the tube socket 130 abut against the hooks 146, the hooking structures 145 are pushed by the tube socket 130 to rotate about the pivotally connecting portions 148 relative to the machine body 141, such that the hooks 146 of the hooking structures 145 can overcome the edge of the tube socket 130 and buckle within the grooves G of the tube socket 130. In this way, the tube socket 130 can be fixed relative to the machine body 141 of the buckling device 140. This means the infusion pipe 200 can also be fixed relative to the machine body 141 of the buckling device 140, and the infusion pipe 200 penetrating through the tube socket 130 can maintain within the first subsidiary through hole H11 of the fixture body 110. At this point, the infusion pipe 200 is suitable for a medical fluid (not shown) to flow therein.

In practical applications, the first end 111 of the fixture body 110 is a holding portion, such that the user can hold it with fingers, and then insert the fixture body 110 into the mounting hole H of the machine body 141. To be specific, a dimension of the first end 111 as the holding portion along the second direction D2 is larger than a dimension of the second through hole H2. Therefore, the tube socket 130 cannot pass through the first end 111 of the fixture body 110 and thus cannot be separated from the fixture body 110. On the contrary, when the user holds the first end 111 as the holding portion by fingers, and then inserts the fixture body 110 into the mounting hole H of the machine body 141, the tube socket 130 is pushed by the first end 111 as the holding portion to move towards the mounting hole H, such that the tube socket 130 can abut against the hooks 146 of the hooking structures 145.

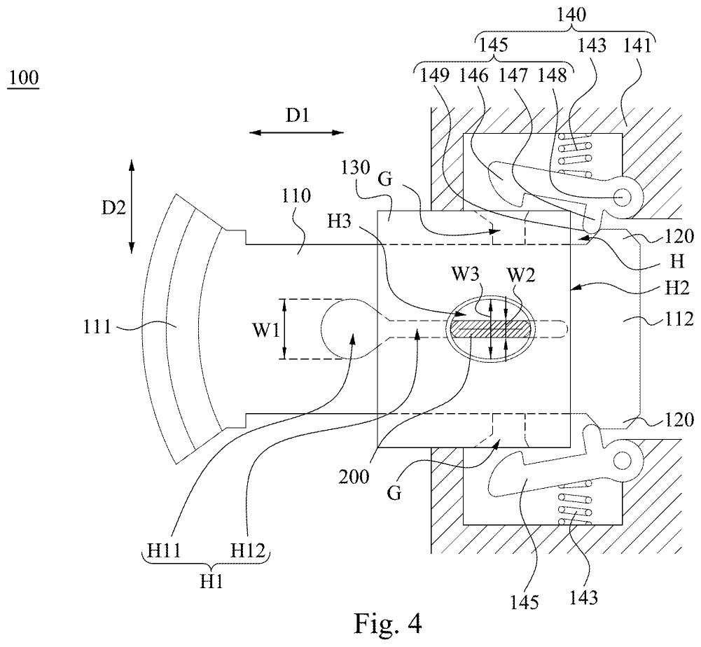

Reference is made to FIG. 4. FIG. 4 is a partial sectional view of the fixture body 110 and the tube socket 130 of FIG. 1 being pulled from a buckling device 140, in which the third through hole H3 of the tube socket 130 corresponds to the second subsidiary through hole H12 of the fixture body 110. As shown in FIG. 4, since the hooks 146 of the hooking structures 145 buckle within the grooves G of the tube socket 130 to fix the tube socket 130 relative to the machine body 141 of the buckling device 140, when the user pulls out the fixture body 110 from the buckling device 140 with fingers holding the first end 111 as the holding portion, the fixture body 110 slides relative to the tube socket 130, such that the third through hole H3 of the tube socket 130 moves to the second subsidiary through hole H12 corresponding to the fixture body 110, and the infusion pipe 200 penetrating through the tube socket 130 switches from the first subsidiary through hole H11 of the fixture body 110 to the second subsidiary through hole H12.

As mentioned above, the first width W1 of the first subsidiary through hole H11 is larger than the second width W2 of the second subsidiary through hole H12. Thus, when the infusion pipe 200 switches from the first subsidiary through hole H11 to the second subsidiary through hole H12, the cross-section of the infusion pipe 200 will be flattened, such that the inner walls of the infusion pipe 200 contact with each other. Thus, the medical fluid can no longer flow through the infusion pipe 200.

In addition, after the infusion pipe 200 switches from the first subsidiary through hole H11 to the second subsidiary through hole H12, the first protruding pieces 120 abut against the second protruding pieces 147 of the hooking structures 145 in a direction pointing an outside of the machine body 141. Thus, the hooking structures 145 rotate about the pivotally connecting portions 148 relative to the machine body 141, such that the hooks 146 buckling within the grooves G are separated from the tube socket 130. In this way, the tube socket 130 is not fixed by the hooks 146 anymore and can be, together with the fixture body 110, separated from the buckling device 140.

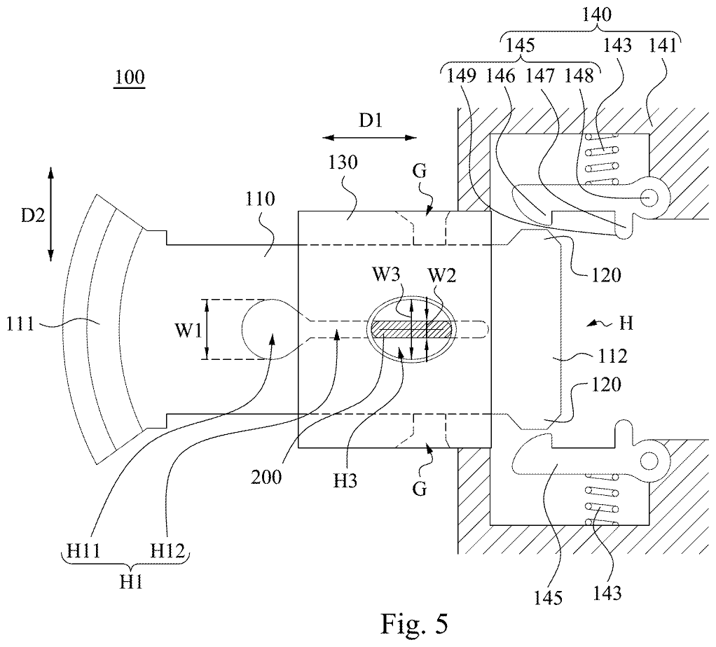

Reference is made to FIG. 5. FIG. 5 is a partial sectional view of the fixture body 110 and the tube socket 130 of FIG. 1 being pulled from a buckling device 140, in which the fixture body 110 and the tube socket 130 are already separated from the buckling device 140. As shown in FIG. 5, after the tube socket 130, together with the fixture body 110, is separated from the buckling device 140, the infusion pipe 200 maintains within the second subsidiary through hole H12 of the fixture body 110. In other words, after the infusion pipe 200 is separated from the buckling device 140, the infusion pipe 200 maintains within the second subsidiary through hole H12 in a status of being flattened. Thus, the medical fluid cannot flow through the infusion pipe 200.

As shown in FIGS. 2-5, the first protruding pieces 120 are symmetrical with each other. In other words, the fixture body 110 and the first protruding pieces 120 are symmetrical structures. This means, when the user inserts the fixture body 110 together with the first protruding pieces 120 into the buckling device 140 or pulls out the fixture body 110 together with the first protruding pieces 120 from the buckling device 140, the forces that the second protruding pieces 147 exert on the fixture body 110 when abutting against the first protruding pieces 120 are also symmetrical. In this way, the process to insert the fixture body 110 together with the first protruding pieces 120 into the buckling device 140 or pulls out the fixture body 110 together with the first protruding pieces 120 from the buckling device 140 becomes more stable. This means, the process to install the infusion pipe 200 to the buckling device 140 or to separate the infusion pipe 200 from the buckling device 140 also becomes more stable. In addition, the risk of damage to the fixture body 110, the first protruding pieces 120 and the hooking structures 145 due to uneven force exertion is also reduced.

Furthermore, before the infusion pipe 200 is separated from the buckling device 140, the infusion pipe 200 is already flattened within the second subsidiary through hole H12 of the fixture body 110. Thus, the medical fluid is prevented from flowing through the infusion pipe 200. Moreover, since the operation described above is carried out in a mechanical manner, the reliability and safety of the operation can be effectively guaranteed.

In conclusion, when compared with the prior art, the aforementioned embodiments of the present disclosure have at least the following advantage:

(1) Since the fixture body and the first protruding pieces are symmetrical structures, when the user inserts the fixture body together with the first protruding pieces into the buckling device or pulls out the fixture body together with the first protruding pieces from the buckling device, the forces that the second protruding pieces exert on the fixture body when abutting against the first protruding pieces are also symmetrical. In this way, the process to install the infusion pipe to the buckling device or to separate the infusion pipe from the buckling device becomes more stable. In addition, the risk of damage to the fixture body, the first protruding pieces and the hooking structures due to uneven force exertion is also reduced.

(2) Before the infusion pipe is separated from the buckling device, the infusion pipe is already flattened within the second subsidiary through hole of the fixture body. Thus, the medical fluid is prevented from flowing through the infusion pipe. Moreover, since the operation described above is carried out in a mechanical manner, the reliability and safety of the operation can be effectively guaranteed.

Although the present disclosure has been described in considerable detail with reference to certain embodiments thereof, other embodiments are possible. Therefore, the spirit and scope of the appended claims should not be limited to the description of the embodiments contained herein.

It will be apparent to the person having ordinary skill in the art that various modifications and variations can be made to the structure of the present disclosure without departing from the scope or spirit of the present disclosure. In view of the foregoing, it is intended that the present disclosure cover modifications and variations of the present disclosure provided they fall within the scope of the following claims.

What is claimed is:

1. An infusion apparatus, comprising:
a fixture body having a first end and a second end opposite to the first end along a first direction, the fixture body having a first through hole extending along the first direction, the first through hole comprising a first subsidiary through hole and a second subsidiary through hole communicating with the first subsidiary through hole, the first subsidiary through hole and the second subsidiary through hole arranging along the first direction, a first width of the first subsidiary through hole along a second direction is larger than a second width of the second subsidiary through hole along the second direction, the fixture body being disposed with a pair of first protruding pieces, the first protruding pieces being located at two opposite sides of the second end along the second direction, the second direction being perpendicular with the first direction;
a tube socket having a second through hole and a third through hole, the fixture body penetrating through the second through hole and being slidable relative to the tube socket along the first direction, the tube socket further having a pair of grooves located at two opposite sides of the tube socket along the second direction, wherein when the tube socket slides relative to the fixture body, the third through hole is able to switch between a first position corresponding to the first subsidiary through hole and a second position corresponding to the second subsidiary through hole; and
a buckling device, comprising:
a machine body having a mounting hole configured to allow the fixture body and the tube socket to penetrate into along the first direction; and
a pair of hooking structures disposed on the machine body and located at two opposite sides of the mounting hole along the second direction, each of the hooking structures having a hook and a second protruding piece, the hooks being configured to buckle within the grooves, a shortest distance between the second protruding pieces is less than a longest distance between the first protruding pieces.

2. The infusion apparatus of claim 1, wherein the first subsidiary through hole is closer to the first end than the second subsidiary through hole to the first end.

3. The infusion apparatus of claim 2, wherein a third width of the third through hole along the second direction is larger than the second width.

4. The infusion apparatus of claim 3, wherein the first width is equal to the third width.

5. The infusion apparatus of claim 2, wherein the first width of the first subsidiary through hole diminishes towards the second subsidiary through hole.

6. The infusion apparatus of claim 1, wherein the first protruding pieces are symmetrical with each other.

7. The infusion apparatus of claim 1, wherein each of the hooking structures has a pivotally connecting portion pivotally connected with the machine body, each of the second protruding pieces is located between the corresponding pivotally connecting portion and the corresponding hook.

8. The infusion apparatus of claim 1, wherein the buckling device further comprises a pair of elastic elements respectively and elastically connected between the corresponding hooking structure and the machine body.

9. The infusion apparatus of claim 1, wherein each of the second protruding pieces has a curved surface, the curved surfaces face to each other.

10. The infusion apparatus of claim 1, wherein each of the first protruding pieces has a trapezoidal shape.
